(19) United States
(12) Patent Application Publication
(10) Pub. No.: US 2015/0209789 A1
(43) Pub. Date: Jul. 30, 2015

(54) SAMPLE PROCESSING APPARATUS AND AUTOMATIC ANALYZING APPARATUS INCLUDING THE SAME

(71) Applicant: INFOPIA CO., LTD., Anyang-si (KR)

(72) Inventors: Byung Hoon Kho, Yongin-si (KR); Dae Kyung Moon, Namyangju-si (KR); Hyunchul Kim, Anyang-si (KR)

(73) Assignee: INFOPIA CO., LTD., Anyang-si (KR)

(21) Appl. No.: 14/415,543

(22) PCT Filed: Oct. 10, 2013
(86) PCT No.: PCT/KR2013/009071

§ 371 Date: Jan. 16, 2015

(30) Foreign Application Priority Data
Oct. 11, 2012 (KR) 10-2012-0113198

(51) Int. Cl. B01L 7/00; B01L 1/00; B01L 3/00; C12Q 1/68

(52) U.S. Cl. CPC B01L 7/52; C12Q 1/6806; B01L 1/00; B01L 3/523; B01L 2300/0832; B01L 2300/04; B01L 2300/06; B01L 2400/06; B01L 2200/10

(57) ABSTRACT

A sample processing apparatus is for extracting a nucleic acid from a sample and amplifying the nucleic acid, wherein the sample processing apparatus including a housing including a chamber; a valve positioned below the housing; and a polymerase chain reaction (PCR) portion positioned below the valve, wherein the polymerase chain reaction portion performing a polymerase chain reaction.

### TECHNICAL FIELD

The present invention relates to a sample processing apparatus and an automatic analyzing apparatus including the same.

### BACKGROUND ART

A method for detecting a pathogen by extracting a nucleic acid, amplifying the nucleic acid, and performing various detecting reactions is used for various studies, medical uses, and industrial uses. In the method for detecting the pathogen, a process of extracting the nucleic acid, a process of amplifying the nucleic acid, a process of detecting the pathogen, and so on should be performed. In each of the processes, various kinds of reactants should be used.

Since several processes using different reactants should be performed in sequence, the processes are complex and it takes a long time for the processes. Particularly, an apparatus for extracting the nucleic acid is different from an apparatus for amplifying the nucleic acid and detecting the pathogen, processes are complex and it takes a long time for the processes.

### DISCLOSURE

### Technical Problem

It is an object of an embodiment of the invention to provide a sample processing apparatus for automatically processing several processes necessary to process a sample and to detect a pathogen and to provide an automatic analyzing apparatus including the sample processing apparatus.

### Technical Solution

A sample processing apparatus is for extracting a nucleic acid from a sample and amplifying the nucleic acid, wherein the sample processing apparatus including a housing including a chamber; a valve positioned below the housing; and a polymerase chain reaction (PCR) portion positioned below the valve, wherein the polymerase chain reaction portion performing a polymerase chain reaction.

An automatic analyzing apparatus according to an embodiment includes a sample processing apparatus for extracting a nucleic acid from a sample and amplifying the nucleic acid; and an apparatus portion where the sample processing apparatus is installed, wherein apparatus portion including a driving member for driving the sample processing apparatus, a heating member for heating the sample processing apparatus, and a detecting member for determining existence and nonexistence of a pathogen from the nucleic acid amplified from the sample processing apparatus. The sample processing apparatus includes: a housing including a chamber; a valve positioned below the housing; and a polymerase chain reaction (PCR) portion positioned below the valve, wherein the polymerase chain reaction portion performing a polymerase chain reaction.

### Advantageous Effects

According to an embodiment of the invention, a housing, a valve, and a polymerase chain reaction (PCR) portion where a polymerase chain reaction is generated are integrally coupled, and thus, a structure can be simplified. In this instance, the housing and the PCR portion are coupled while interposing the valve, and thus, the valve can be rotated. Accordingly, by a rotation of the valve, several processes of extracting a nucleic acid from a sample, amplifying the nucleic acid, and detecting a pathogen can be automatically performed in sequence.

That is, according to the embodiment, processing a sample and detecting a pathogen can be automated by a simple structure.

### BRIEF DESCRIPTION OF DRAWINGS

FIG. 1 is a perspective view of a sample processing apparatus according to an embodiment of the invention.

FIG. 2 is an exploded perspective view of the sample processing apparatus shown in FIG. 1.

FIG. 3 is a perspective view of a valve of the sample processing apparatus shown in FIG. 1.

FIG. 4 is a partial cross-sectional view of the sample processing apparatus shown in FIG. 1.

FIG. 5 is a plan view of a housing of the sample processing apparatus shown in FIG. 1.

FIG. 6 is a cross-sectional view taken in a line VI-VI of FIG. 5.

FIG. 7 is a partial cross-sectional view of a sample processing apparatus according to a modified embodiment of the invention.

FIG. 8 is a partial cross-sectional view of a sample processing apparatus according to another modified embodiment of the invention.

FIG. 9 is a perspective view of a polymerase chain reaction (PCR) portion of the sample processing apparatus shown in FIG. 1.

FIG. 10a and FIG. 10b are cross-section views taken in a line of X-X of FIG. 9.

FIG. 11 is a perspective view of an automatic analyzing apparatus according to an embodiment of the invention.

FIG. 12 is a schematically cross-sectional view of the automatic analyzing apparatus shown in FIG. 11.

FIG. 13a to FIG. 13l are views for describing an operation of the sample processing apparatus according to the embodiment.

FIG. 14 is a sectional perspective view of a housing of an automatic analyzing apparatus according to a modified embodiment.

FIG. 15 is a perspective view of a PCR portion of the automatic analyzing apparatus according to the modified embodiment.

### DETAILED DESCRIPTIONS OF THE EMBODIMENTS

Reference will now be made in detail to the preferred embodiments of the present invention, examples of which are illustrated in the accompanying drawings. However, it will be understood that the present invention should not be limited to the embodiments and may be modified in various ways.

In the drawings, to clearly and briefly explain the present invention, illustration of elements having no connection with the description is omitted, and the same or extremely similar elements are designated by the same reference numerals throughout the specification. In addition, in the drawings, for more clear explanation, the dimensions of elements, such as thickness, width, and the like, are exaggerated or reduced, and thus the thickness, width, and the like of the present invention are not limited to the illustration of the drawings.

In the entire specification, when an element is referred to as “including” another element, the element should not be understood as excluding other elements so long as there is no special conflicting description, and the element may include at least one other element. In addition, it will be understood that, when an element such as a layer, film, region or substrate is referred to as being “on” another element, it can be directly on the other element or intervening elements may also be present. On the other hand, when an element such as a layer, film, region or substrate is referred to as being “directly on” another element, this means that there are no intervening elements therebetween.

Hereinafter, a sample processing apparatus and an automatic analyzing apparatus including the same according to embodiments of the present invention will be described in detail with reference to the accompanying drawings.

A sample processing apparatus according to an embodiment automatically performs a process of extracting a nucleic acid from a sample (or a specimen) and a process of amplifying the nucleic acid to be used to detect a pathogen and so on.

In this instance, the sample indicates all samples including the nucleic acid. The sample may include a virus, a microorganism, a cell, a tissue of an animal or a plant, an organ of an animal or a plant, a body fluid, and so on. For example, the sample is gathered from an organ such as a spleen, the other body fluid component or tissue in order to detect the pathogen. The sample may include a specific disease tissue, a tissue having a biomarker, a sample of predilection sites of a pathogenic microorganism (for example, blood, a tissue, sputum, urine, feces, and so on), a sample proliferated by a cell culture, a sample of the natural world, and so on. The sample may be obtained by known various methods.

The nucleic acid may be a genetic material including a polynucleotide. The nucleic acid may be classified into a deoxyribonucleic acid (DNA) and a ribonucleic acid (RNA).

The sample processing apparatus is applied to an automatic analyzing apparatus including a detecting member for detecting the pathogen and so on. A process of automatically extracting the nucleic acid from the sample, a process of amplifying the nucleic acid, a process of detecting the pathogen, and so on can be performed by the automatic analyzing apparatus.

Hereinafter, the sample processing apparatus will be described, and then, the automatic analyzing apparatus including the sample processing apparatus will be described. After that, operations of them will be described in detail.

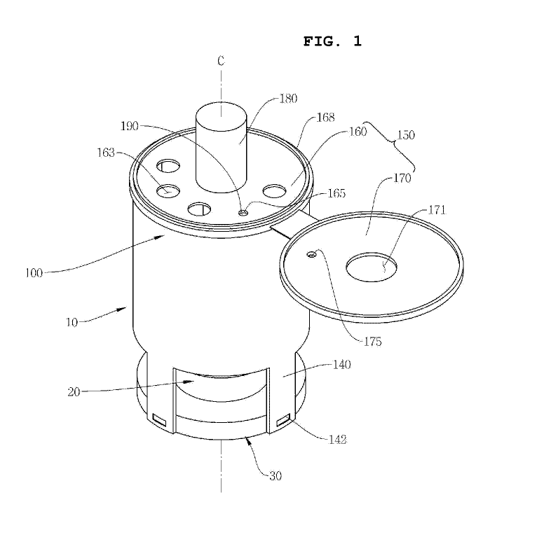

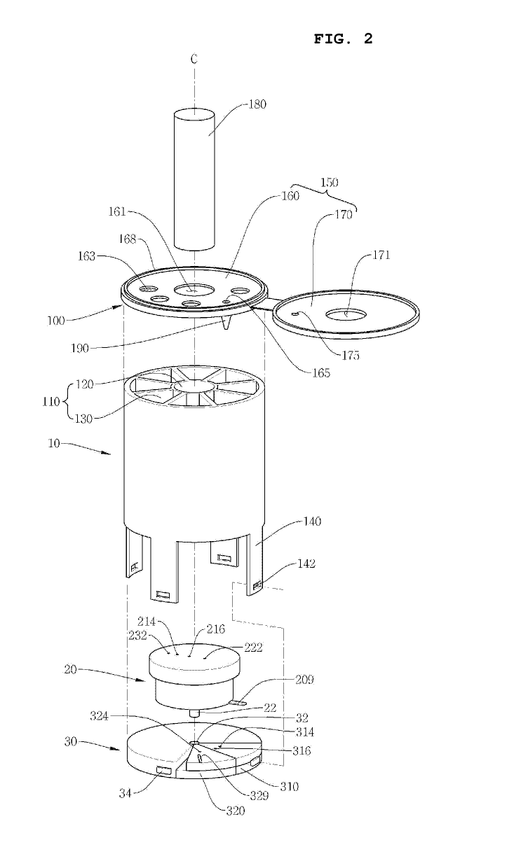

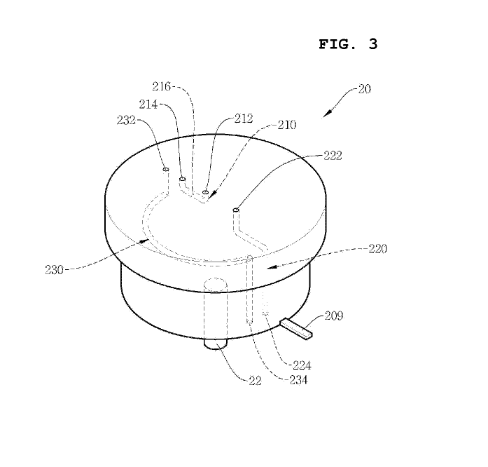

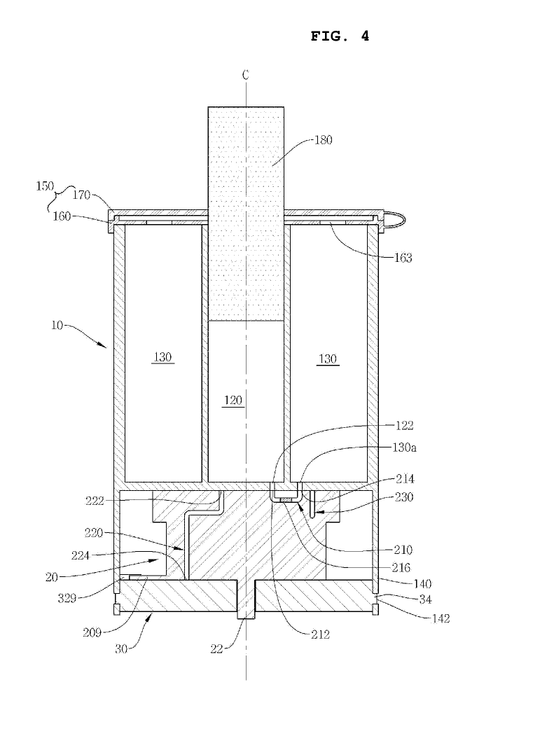

FIG. 1 is a perspective view of a sample processing apparatus according to an embodiment of the invention, and FIG. 2 is an exploded perspective view of the sample processing apparatus shown in FIG. 1. Also, FIG. 3 is a perspective view of a valve of the sample processing apparatus shown in FIG. 1, and FIG. 4 is a partial cross-sectional view of the sample processing apparatus shown in FIG. 1.

Referring to FIG. 1 and FIG. 2, a sample processing apparatus 100 according to an embodiment includes a housing having a plurality of chambers 110, a valve 20 positioned below the housing 10, and a polymerase chain reaction (PCR) portion 30 positioned below the valve 20.

The housing 10 may have a shape having an upper portion that is open and a bottom surface. For example, the housing 10 may have a substantially cylindrical shape having a planar shape of a circular shape. However, the present invention is not limited thereto, and thus, the housing 10 may have various shapes. The housing 10 may be made of one or more of various materials for receiving and supporting materials inside the housing 10. For example, the housing 10 may be made of a plastic.

The housing 10 may include a plurality of chambers 110 for performing several processes of eluting the nucleic acid obtained by a lysis of the sample in sequence inside the housing 10. The plurality of chambers 110 may include a fluid flow chamber 120 positioned at a central portion, and a reaction chamber 130 positioned at a peripheral portion of the fluid flow chamber 120. The reaction chamber 130 may include a plurality of reaction chambers 130 for performing several processes of processing the sample in sequence. The fluid flow chamber 120 for supplying a fluid to one of the reaction chambers 130 or the PCR portion 30 in each of the processes or for receiving the fluid from one of the reaction chambers 130 or the PCR portion 30 in each of the processes is positioned at the central portion. Thus, the moving line of the fluid flow can be minimized.

A plurality of holes for the fluid flow are formed at a bottom surface of fluid flow chamber 120 and the reactant chambers 130. This will be described in detail with reference to FIG. 3 and FIG. 4 later.

Extended portions 140 are formed at the bottom surface of the housing 10. The extended portions 140 are extended from the bottom surface of the housing 10 to be separated from each other and form a side surface or side surfaces of the housing 10. The extended portions 140 crosses the valve 20 and reaches a side surface of the PCR portion 30 in the state that the valve 20 is interposed between the housing 10 and the PCR portion 30.

A first coupled portion 142 being coupled to the PCR portion 30 is formed at a portion of the extended portion 140 corresponding to the PCR portion 30. The first coupled portion 142 of the extended portions 140 is coupled to a second coupled portion 34 formed at the side surface of the PCR portion 30. Thus, the housing 10 and the PCR portion 30 can be integrally fixed and the valve 20 can be rotatably positioned between the housing 10 and the PCR portion 30.

It is exemplified that a number of the extended portions 140 is four in the drawings. However, the invention is not limited thereto. It is sufficient that the extended portions 140 can integrally fix the housing 10 and the PCR 30 while interposing the valve 20. Thus, a number of the extended portions 140 may be varied. Accordingly, the number of the extended portions 140 may be two or more.

A cover portion 150 for covering the reaction chambers 130 may be positioned on the housing 10. An opening 161 and 171 for opening the fluid flow chamber 120 is formed at a central portion of the cover portion 150. A fluid moving member 180 may be positioned inside the fluid flow chamber 120 through the opening 161 and 171. The fluid moving member 180 moves up and down to control the fluid flow inside the fluid flow chamber 120. For example, the fluid moving member 180 may be a plunger or a piston.

In the embodiment, the cover portion 150 may include a first cover portion 160 and a second cover portion 170.

The first cover portion 160 may be fixed at an upper edge of the housing 10. For example, the first cover portion 160 may surround the upper edge of the housing 10 and be fixed to the upper edge of the housing 10. Also, a fixing protruded portion 168 protruding to the outside is formed along the edge of the first cover portion 160 at the upper surface of the first cover portion 160.

In this instance, second openings 163 and a third opening 165, as well as the opening 161, may be formed at the first cover portion 160. Each of the second openings 163 correspond to each of the reaction chambers 130. The ultrasonic member 190 is inserted into the third opening 165. The ultrasonic member 190 and a method for fixing the ultrasonic 190 will be described later.

The second cover portion 170 is fixed on the first cover portion 160 on the first cover portion 160. For example, the second cover portion 170 has an edge protruding to surround the fixing protruded portion 168 formed at the first cover portion 160. Accordingly, the edge of the second cover portion 170 is positioned at an outside of the first cover portion 160, and the second cover portion 170 is fixed on the first cover portion 160.

In this instance, a third opening 175, as well as the opening 171, may be formed at the second cover portion 170. The ultrasonic member 190 is inserted into the third opening 175. The second cover portion 170 covers all portions of the reaction chambers 130.

In the embodiment, the cover portion 150 has a double structure having the first cover portion 160 and the second cover portion 170. Thereby, the reaction chambers 130 are open through the second openings 163 when the second cover portion 170 is open. Thus, when materials are supplied to each of the reaction chambers 130 and materials are discharged from each of the reaction chambers 130, the reaction chambers 130 can be open by opening the second cover portion 170. When the materials are not supplied or are not discharged, the reaction chambers 130 can be closed by covering the second cover portion 170 on the first cover portion 160.

In this instance, a part of the second cover portion 170 and a part of the second cover portion 160 are foldably connected to each other. Accordingly, the opening and closing of the second cover portion 170 can be easily performed, and the second cover 170 is prevented from being lost when the second cover portion 170 is open.

The cover portion 150 may include one or more of various materials so that the materials of the plurality of the chambers 110 are not discharged to the outside. For example, the cover portion 150 may be made of plastic.

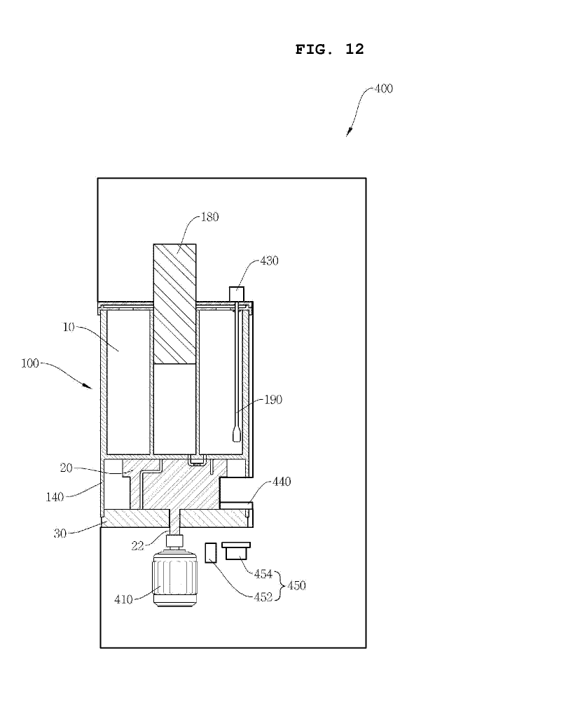

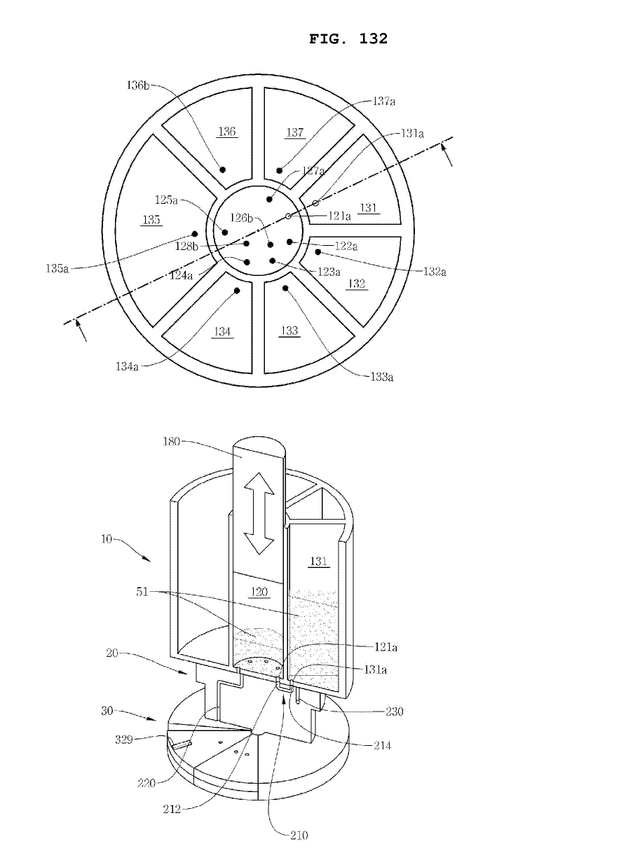

The valve 20 for controlling the fluid flow to the plurality of the chambers 110 of the housing 10 and the PCR portion 30 is positioned between the housing 10 and the PCR portion 30. The valve 20 may be connected to a rotation driving member (reference numeral 410 of FIG. 12) through a connection member 22 penetrating the central portion of the PCR portion 30 and extended to the outside. Also, the valve 20 can be freely rotated clockwise or counterclockwise by the rotation driving member 410. In the embodiment, the valve 20 may have a substantially discus shape having a planar shape of a circular shape.

Referring to FIG. 3, a plurality of channels 210, 220, and 230 are formed inside the valve 20. Through the channels 210, 220, and 230, the fluids can flow between the chambers 110 or between the chamber 110 and the PCR portion 30. An upper surface of the valve 20 may be in close contact with the housing 10 (more particularly, a bottom surface of the housing 10), and a lower surface of the valve 20 may be in close contact with the PCR portion 30 (more particularly, an upper surface of the PCR portion 30). Thereby, when the plurality of the channels 210, 220, and 230 are connected to the holes of the chambers 110 and the outlet of the PCR portion 30, the fluid can be prevented from being discharged to the outside. The plurality of channels 210, 220, and 230 penetrate the upper surface of the valve 20 and the lower surface of the valve 20. And thus, additional channels are not included in the housing 10. Accordingly, a structure can be simplified and the fluid can smoothly flow.

An area of the lower surface of the valve 20 is smaller than an area of the upper surface of the valve 20. Then, the upper surface of the PCR portion 30 can be exposed with a large area, and thus, a heating member (reference numeral 440 of FIG. 12) can be widely positioned on the upper surface of the PCR portion 30. For example, the valve 20 may include an upper portion having a first diameter in a plan view and a lower portion having a second diameter smaller than the first diameter in the plan view. Also, a planar area of the valve 20 is smaller than a planar area of the housing 10 and the PCR portion 30, and thus, the valve 20 can freely rotate between the housing 10 and the PCR portion 30, regardless to the fixing structure of the housing 10 and the PCR portion 30.

More particularly, the plurality of channels 210, 220, and 230 include a first channel 210 and a second channel 220. The first channel 210 connects the fluid flow chamber 120 and each of the reaction chambers 130. The second channel 220 connects the fluid flow chamber 120 and the PCR portion 30. Also, the plurality of channels 210, 220, and 230 may include a third channel 230 for connecting any one of the reaction chambers 130 and the PCR portion 30.

The first channel 210 is interposed between a first outlet 212 and a second outlet 214. The first outlet 212 is formed at the upper surface of the valve 20 to be connected to the fluid flow chamber 120. The second outlet 214 is formed at the upper surface of the valve 20 to be connected to the reaction chamber 130.

In this instance, the first outlet 212 corresponds to the fluid flow chamber 120 and a distance between the first outlet 212 and a central axis C of the valve 20 is relatively small. The second outlet 214 corresponds to each of the reaction chambers 130, and a distance between the second outlet 214 and the central axis C of the valve 20 is relatively large. Also, the first outlet 212 and the second outlet 214 may be positioned in parallel in a diameter crossing a center of the valve 20. Thus, a path of the first channel 210 formed between the first outlet 212 and the second outlet 214 can be simplified.

A first filter 216 for collecting the nucleic acid in the sample is formed inside the first channel 210. The first filter 216 may be made of one or more of various materials for known various materials being able to collect the nucleic acid. For example, a porous material of a glass fiber may be used for the first filter 216.

The second channel 220 is interposed between a third outlet 222 and a fourth outlet 224. The third outlet 222 is formed at the upper surface of the valve 20 to be connected to the fluid flow chamber 120. The fourth outlet 224 is formed at the lower surface of the valve 20 to be connected to the PCR portion 30. In this instance, the third outlet 222 is closer to the first outlet 212 than the central axis C of the valve 20. However, the invention is not limited thereto. Therefore, it is sufficient that the third outlet 222 is positioned at a separated position from the first outlet 212, and thus, the position of the third outlet 222 is not limited.

The third channel 230 is interposed between a fifth outlet 232 and a sixth outlet 234. The fifth outlet 232 is formed at the upper surface of the valve 20 to be connected to the reaction chamber 130. The sixth outlet 234 is formed at the lower surface of the valve 20 to be connected to the PCR portion 30. In the instance, a distance between the fourth outlet 224 and the central axis C of the valve 20 is the same as a distance between the sixth outlet 234 and the central axis C of the valve so that the fluid can smoothly flow to the PCR portion 30. This will be described in detail later when the PCR portion 30 will be described. The fifth outlet 232 may be farther from the central axis C of the valve 20 than the second outlet 214.

A PCR moving portion 209 may be formed at the lower surface of the valve 20 to protrude so that the PCR moving portion 209 may be engaged with an engaged portion 329 formed at the upper surface of the PCR portion 30. Various structures being able to be engaged with each other and to rotate a part of the PCR portion 30 according to the rotation of the valve may be applied to the engaged portion 329 and the PCR moving portion 209. This will be described in more detail.

For example, the valve 20 may be made of a plastic. However, the invention is not limited thereto, and the valve 20 may be made of the other various materials.

Referring to FIG. 1 and FIG. 2 again, the PCR portion 30 may be positioned below the valve 20. The PCR portion 30 performs a polymerase chain reaction of the nucleic acid collected at the housing 10 and amplifies the DNA. In the embodiment, the PCR portion 30 may have a substantially discus shape having a planar shape of a circular shape.

A penetration hole 32 is formed at a central portion of the PCR portion 30. The connection member 22 connected to the valve 20 may penetrate through the penetration hole 32. Also, the second coupled portions 34 coupled to the first coupled portion 142 of the extended portions 140 of the housing 10 are formed at the side surface of the PCR portion 30. The second coupled portion 34 may have one of various structures being able to be coupled to the first coupled portion 142. For example, in the embodiment, the second coupled portion 34 may be a protruded portion formed at the side surface of the PCR portion 30 so that the second coupled portion 34 can be inserted to the first coupled portion 142 having a form of a hole (or a recess or a groove).

The valve 20 is positioned on the PCR portion 30 in a state that the connection member 22 of the valve 20 penetrates through the penetration hole 32 of the PCR portion 30. And then, the housing 10 is positioned on the valve 20 and the PCR portion 30. After that, the second coupled portion 34 (that is the protruded portion) of the PCR portion 30 is inserted into and then fixed to the first coupled portion 142 (that is, the hole) of the extended portions 140. Accordingly, the housing 10, the valve 20, and the PCR portion 30 can be integrally coupled. In this instance, the housing 10 and the PCR portion 30 are fixed by the first and second coupled portions 142 and 34, while the valve 20 is not fixed to the housing 10 and the PCR portion 30. Therefore, when the valve 20 is connected to the rotation driving member 410 through the connection member 22, the valve 20 can rotate.

In the embodiment, by the insertion of the protruded portion into the hole, the housing 10, the valve 20, and the PCR portion 30 can be integrally coupled. Thus, the housing 10, the valve 20, and the PCR portion 30 can be coupled by a simple process. In this instance, a planar shape of each of the housing 10, the valve 20, and the PCR portion 30 may be a circular shape. Then, the housing 10, the valve 20, and the PCR portion 30 can be easily integrated, and the valve 20 can smoothly rotate.

In the above descriptions, it is exemplified that the hole is the first coupled portion 142 and the protruded portion is the second coupled portion 34. However, the invention is not limited thereto. The first coupled portion 142 may be the protruded portion, and the second coupled portion 34 may be the hole. Also, the other various coupled members may be used.

In the embodiment, the PCR portion 30 includes a first PCR portion 310 and a second PCR portion 320 having reactions spaces of different shapes in order to perform a two-step PCR. This will be described later. For example, the PCR portion 30 may be formed of a plastic. However, the invention is not limited thereto. The PCR portion 30 may be made of one or more of the other materials.

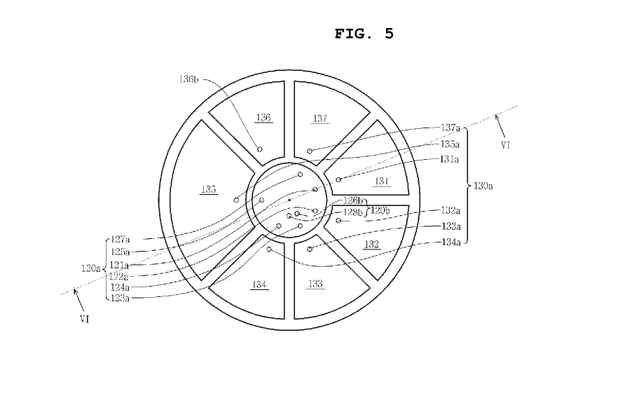

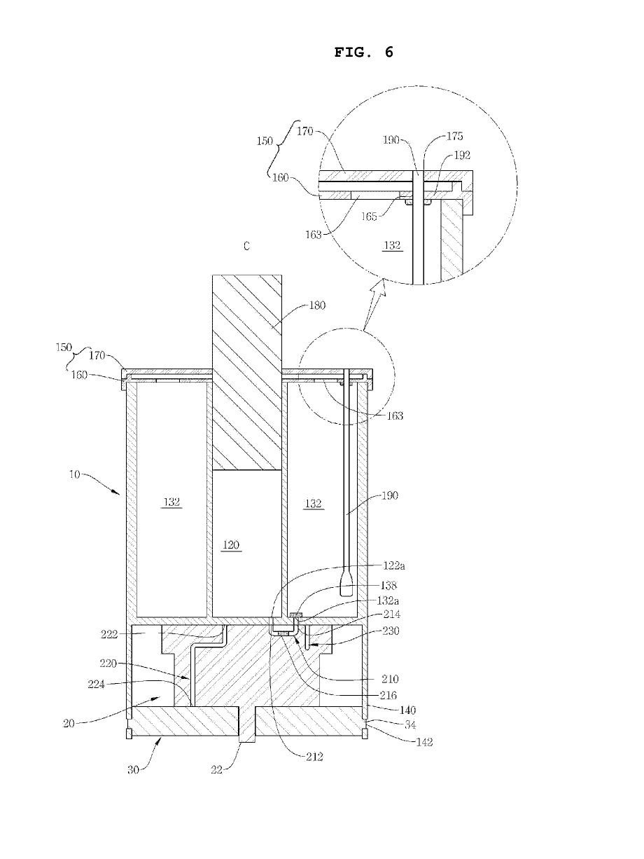

Hereinafter, structure of the housing 10 and the PCR portion 30 will be described in more detail. The plurality of the chambers 110 in the housing 10 will be described in more detail with reference to FIG. 5 and FIG. 6, and then, the PCR portion 30 will be described in more detail.

FIG. 5 is a plan view of the housing of the sample processing apparatus shown in FIG. 1, and FIG. 6 is a cross-sectional view taken in a line VI-VI of FIG. 5.

Referring to FIG. 4 and FIG. 5, the plurality of the chambers 110 including the fluid flow chamber 120 and the reaction chambers 130 are formed at the inside of the housing 10. The reaction chambers 130 will be described, and then, the fluid flow chamber 120 will be described.

The plurality of the reaction chambers 130 may be positioned at the peripheral portion of the fluid flow chamber 120. For example, the plurality of the reaction chambers 130 extend toward the outside from the fluid flow chamber 120. When the plurality of the reaction chambers 130 have the above structure, a space inside the housing 10 can be effectively used. Thus, all spaces necessary to an analysis can be included, and also, a size of the sample processing apparatus can be reduced.

Each of the reaction chambers 130 has a material being able to perform a lysis of a cell of the sample or perform an elution of the nucleic acid obtained from the lysis. The reaction chambers 130 have different inner volumes with consideration of amounts of solutions or materials necessary for the reactions, reaction spaces, and so on. However, the invention is not limited thereto. The reaction chambers 130 may have the same inner volumes.

For example, in the embodiment, the reaction chambers 130 may include a binding chamber 131, a lysis chamber 132, washing chambers 133 and 134, an elution chamber 135, the discard chamber 136 and a dilution chamber 137. However, the invention is not limited thereto. Thus, one or more of the reaction chambers 130 may be omitted, or an additional chamber may be added. Accordingly, various modifications are possible.

In the embodiment, the binding chamber 131, the lysis chamber 132, the washing chambers 133 and 134, the elution chamber 135, the discard chamber 136, and the dilution chamber 137 are positioned clockwise in sequence so that several reactions are performed as the valve 20 rotates clockwise. Then, the rotation of the valve 20 can be minimized, and energy necessary to drive the valve 20 can be minimized. However, the present invention is not limited thereto. Therefore, the valve 20 may rotate counterclockwise and the reaction chambers 130 may be positioned counterclockwise in sequence, contrary to the embodiment. Also, the reaction chambers 130 may be arranged in no particular order. Thus, various modifications are possible.

The binding chamber 131 contains a binding buffer or the remained binding buffer after the usage. The binding buffer includes a component being able to effectively collect the nucleic acid at the first filter 216. The binding buffer passes through the first filter 216 in the first channel 210 and provides an environment that the nucleic acid can be effectively collected at the first filter 216. One or more of known various materials may be used as the binding buffer. For example, at least one chaotropic salt selected from a group of consisting of guanidine-HCl, guanidine-SCN, and NaI may be used for the binding buffer.

A first hole 131a to be connected to the first channel 210 formed at the valve 20 is formed at a bottom surface of the binding chamber 131. The first channel 210 for connecting the fluid flow chamber 120 and each of the reaction chambers 130 is connected to the first hole 131a. More particularly, the first hole 131a is formed at a position corresponding to the second outlet 214 formed at the upper surface of the valve 20 to correspond to each of the reaction chambers 130. That is, a distance between the central axis C of the housing 10 and the first hole 131a is the same as a distance between the central axis C of the valve 20 and the second outlet 214. Then, when the second outlet 214 of the valve 20 coincides with the first hole 131a by the rotation, the first channel 210 is connected to the binding chamber 131.

The lysis chamber 132 contains a lysis buffer for the lysis of the sample. One or more of known various materials may be used as the lysis buffer. For example, the lysis buffer may include a chaotropic agent such as guanidinium salt (for example, guanidinium thio cyanate), a chelating agent such as ethylenediaminetetraacetic acid (EDTA), a buffer salt such as trihidroxymethylaminomethane (Tris-HCl), and so on. Also, the lysis buffer may include a nonionic surfactant agent. A nonionic surfactant agent of a polyethyleneglycol type or a nonionic surfactant agent of a polyhydric-alcohols type may be used. For example, Triton X-100, Tween (adducts of ethylene oxide of sorbitan ester) or 2-mercaptoethanol may be used, preferably, Triton X-100 may be used. The lysis buffer may be non-acid, for example, neutral or alkalic. However, the invention is not limited thereto, and the lysis buffer may include one or more of various materials.

A first hole 132a to be connected to the first channel 210 formed at the valve 20 is formed at a bottom surface of the lysis chamber 132. The first channel 210 for connecting the fluid flow chamber 120 and each of the reaction chambers 130 is connected to the first hole 132a. More particularly, the first hole 132a is formed at a position corresponding to the second outlet 214 formed at the upper surface of the valve 20 to correspond to each of the reaction chambers 130. That is, a distance between the central axis C of the housing 10 and the first hole 132a is the same as a distance between the central axis C of the valve 20 and the second outlet 214. Then, when the second outlet 214 of the valve 20 coincides with the first hole 132a by the rotation, the first channel 210 is connected to the lysis chamber 132.

In this instance, referring to FIG. 6, a second filter 138 is positioned on the first hole 132a of the lysis chamber 132. The second filter 138 is for removing cell debris. The second filter 138 has an area larger than the first hole 132a of the lysis chamber 132 to cover all portions of the first hole 132a. Thereby, when the nucleic acid inside the lysis chamber 132 is discharged through the first hole 132a, the cell debris can be sufficiently filtered by the second filter 138.

The second filter 138 may have filtering holes that the nucleic acid can passes through and the cell debris cannot pass through. A size of the filtering hole is not limited, and the size of the filtering hole may be about 0.2 um to about 50 um. When the size is smaller than about 0.2 um, a part of the DNA may not pass through the filtering holes of the second filter 133. When the size is larger than about 50 un, the cell may pass through the filtering holes of the second filter 138.

In the embodiment, the conventional centrifugal process performed to eliminate the cell debris can be replaced with the second filter 138 on the first hole 132a. Thus, the process can be simplified. Also, problems that may be induced from the cell debris can be prevented at the subsequent processes.

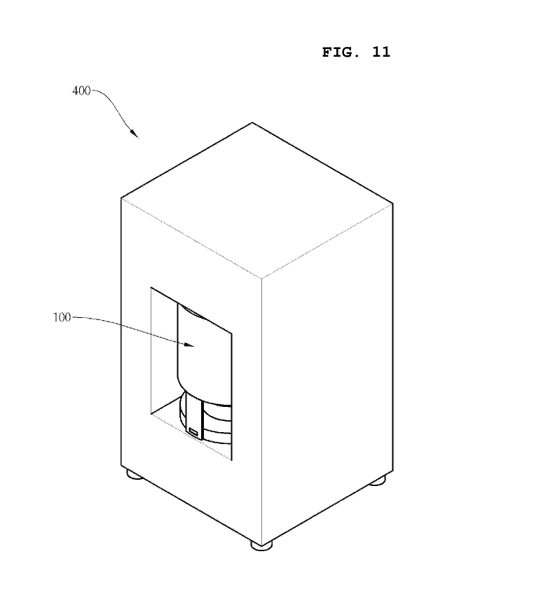

An ultrasonic member 190 is positioned at the cover portion 150 to correspond to the lysis chamber 132. More particularly, the third opening 165 and 175 is formed at the cover portion 150 to correspond to the lysis chamber 132, the ultrasonic member 190 penetrates through the third opening 165 and 175, and thus, the ultrasonic member 190 is positioned inside the lysis chamber 132. An upper portion of the ultrasonic member 190 is exposed through the third opening 165 and 175 to the outside, and the ultrasonic member 190 can be easily connected to an ultrasonic-member driving member (reference numeral 430 of FIG. 12) for providing energy to the ultrasonic member 190 in an automatic analyzing apparatus (reference numeral 400 of FIG. 11).

The ultrasonic member 190 may be fixed to the first cover portion 160 by an adhesive portion 192 so that the connection of the ultrasonic member 190 and the ultrasonic-member driving member 430 is not disturbed. One or more of known various materials may be used as the adhesive portion 192.

However, the invention is not limited thereto. As shown in FIG. 7, the ultrasonic member 190 may penetrate only the third opening 165 of the first cover portion 160, and a connection member 176 (such as a metal) for being connected to the ultrasonic-member driving member 430 may be included at a portion of the second cover portion 170 where the ultrasonic member 190 is in contact. For example, the connection member 176 may be adhered to the third opening 175 of the second cover portion 170 by an adhesive layer 194.

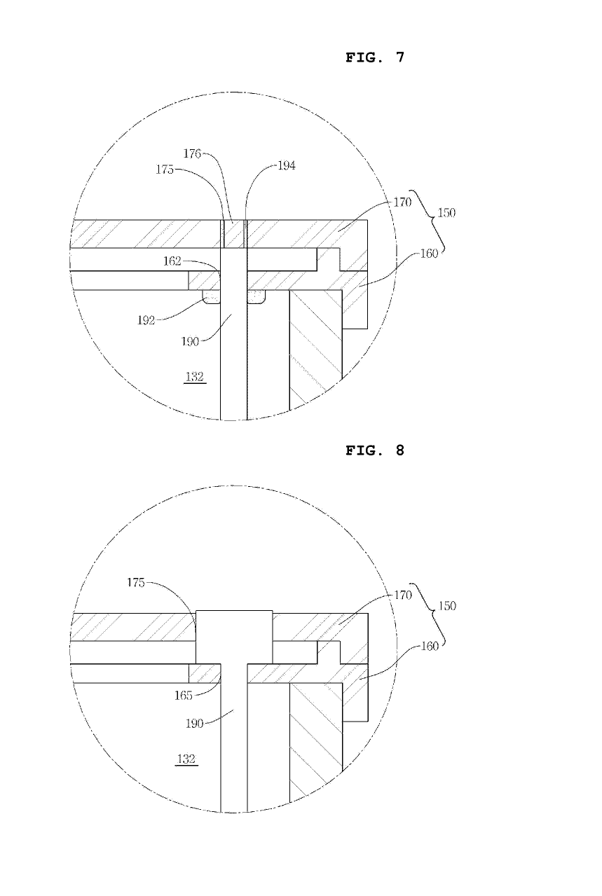

Selectively, as shown in FIG. 8, an area of the third opening 175 of the second cover portion 170 is larger than an area of the third opening 165 of the first cover portion 160. Then, a step is formed at a side surface of the third opening 165 and 175 of the cover portion 150. The ultrasonic member 190 can be stable installed when the ultrasonic member 190 penetrates through the third opening 165 and 175 having the step at the side surface. The fixing structure and method of the ultrasonic member 190 may be variously varied.

The ultrasonic member 190 can facilitate the lysis of the sample by providing the ultrasonic waves to the cell and the lysis buffer during the lysis process. The ultrasonic member 190 has a tip shape having a sharp front end to provide the ultrasonic waves. The types and the structures of the ultrasonic member 190 may be varied.

The washing chambers 133 and 134 contain a washing buffer for washing the first filter 126 or contain the washing buffer and impurities after washing the first filter 126. The washing buffer increases a purity of the target nucleic acid by washing the impurities that may existed along with the nucleic acid at the first filter 126 or a reactant solution (particularly, a chaotropic salt) used for the previous process.

For example, in the embodiment, the washing chambers 133 and 134 include the first and second washing chambers 133 and 134 separately formed, and thus, the impurities existed in the first filter 126 can be effectively eliminated. In another embodiment, the number of the washing chamber may be one.

A first washing buffer for a first-washing may be positioned in the first washing chamber 133. The first washing buffer is not limited if the first washing buffer selectively eliminates the impurities besides the nucleic acid. For example, the first washing buffer may include ethanol, isopropanol, and so on of a concentration of 90% to 100%.

A first hole 133a to be connected to the first channel 210 formed at the valve 20 is formed at a bottom surface of the first washing chamber 133. The first channel 210 for connecting the fluid flow chamber 120 and each of the reaction chambers 130 is connected to the first hole 133a. More particularly, the first hole 133a is formed at a position corresponding to the second outlet 214 formed at the upper surface of the valve 20 to correspond to each of the reaction chambers 130. That is, a distance between the central axis C of the housing 10 and the first hole 133a is the same as a distance between the central axis C of the valve 20 and the second outlet 214. Then, when the second outlet 214 of the valve 20 coincides with the first hole 133a by the rotation, the first channel 210 is connected to the first washing chamber 133.

A second washing buffer for a second-washing may be positioned in the second washing chamber 134. The second washing buffer may be the same as or be different from the first washing buffer. When the second washing buffer is the same as first washing buffer, the second washing buffer can eliminate the impurities remained at the first filter 126 more. The second washing buffer having a different material or a component ratio from that the first washing buffer may be used. When the second washing buffer is the same as the first washing buffer, the second washing buffer can eliminate the impurities more and eliminate the chaotropic salt of the first washing buffer. Accordingly, the nucleic acid can be easily dissolved from the first filter 126 by using the elution buffer during the elution process that is a next step of the second washing. Since alcohol that is one material of the first washing buffer may have an effect of suppressing the PCR reaction, the second washing buffer is used to effectively eliminate the alcohol. For example, ethanol of a concentration of 50˜80% may be included in the second washing buffer.

A first hole 134a to be connected to the first channel 210 formed at the valve 20 is formed at a bottom surface of the second washing chamber 134. The first channel 210 for connecting the fluid flow chamber 120 and each of the reaction chambers 130 is connected to the first hole 134a. More particularly, the first hole 134a is formed at a position corresponding to the second outlet 214 formed at the upper surface of the valve 20 to correspond to each of the reaction chambers 130. That is, a distance between the central axis C of the housing 10 and the first hole 134a is the same as a distance between the central axis C of the valve 20 and the second outlet 214. Then, when the second outlet 214 of the valve 20 coincides with the first hole 134a by the rotation, the first channel 210 is connected to the second washing chamber 134.

The elution chamber 135 contains an elution buffer for eluting the nucleic acid collected at the first filter 126. One or more of various materials being able to dissolve the nucleic acid collected at the first filter 126 may be used as the elution buffer. For example, water, a TE buffer (Tris-Cl, EDTA), or so on may be used as the elution buffer.

A first hole 135a to be connected to the first channel 210 formed at the valve 20 is formed at a bottom surface of the elution chamber 135. The first channel 210 for connecting the fluid flow chamber 120 and each of the reaction chambers 130 is connected to the first hole 135a. More particularly, the first hole 135a is formed at a position corresponding to the second outlet 214 formed at the upper surface of the valve 20 to correspond to each of the reaction chambers 130. That is, a distance between the central axis C of the housing 10 and the first hole 135a is the same as a distance between the central axis C of the valve 20 and the second outlet 214. Then, when the second outlet 214 of the valve 20 coincides with the first hole 135a by the rotation, the first channel 210 is connected to the elution chamber 135.

The discard chamber 136 is connected to the third channel 230 when the elution solution including the nucleic acid is supplied to the PCR portion 30 (particularly, the first PCR 310). Then, an air in the PCR portion 30 can flow to the discard chamber 136, and the elution solution including the nucleic acid can be supplied to the PCR portion 30.

A first hole 136a to be connected to the first channel 210 formed at the valve 20 is formed at a bottom surface of the discard chamber 136. The first channel 210 for connecting the fluid flow chamber 120 and each of the reaction chambers 130 is connected to the first hole 136a. More particularly, the first hole 136a is formed at a position corresponding to the second outlet 214 formed at the upper surface of the valve 20 to correspond to each of the reaction chambers 130. That is, a distance between the central axis C of the housing 10 and the first hole 136a is the same as a distance between the central axis C of the valve 20 and the second outlet 214. Then, when the second outlet 214 of the valve 20 coincides with the first hole 136a by the rotation, the first channel 210 is connected to the discard chamber 136.

In the dilution chamber 137, a diluted mixture is formed by mixing a material treated at the PCR portion 30 (particularly, the PCR portion 310)(hereinafter, referred to as “the first treated material”) and the dilution buffer. One or more of various materials for diluting the first treated material may be used as the dilution buffer with a suitable amount.

For example, an amount of the dilution buffer is 5 times to 15 times of an amount of the first treated material. When the amount of the dilution buffer is smaller than 5 times the amount of the first treated material, the dilution may be not sufficient. When the amount of the dilution buffer is larger than 15 times the amount of the first treated material, the diluted mixture may be excessively large. However, the invention is not limited thereto, and the amount of the dilution buffer may be suitably controlled.

A first hole 137a to be connected to the first channel 210 formed at the valve 20 is formed at a bottom surface of the dilution chamber 137. The first channel 210 for connecting the fluid flow chamber 120 and each of the reaction chambers 130 is connected to the first hole 137a. More particularly, the first hole 137a is formed at a position corresponding to the second outlet 214 formed at the upper surface of the valve 20 to correspond to each of the reaction chambers 130. That is, a distance between the central axis C of the housing 10 and the first hole 137a is the same as a distance between the central axis C of the valve 20 and the second outlet 214. Then, when the second outlet 214 of the valve 20 coincides with the first hole 137a by the rotation, the first channel 210 is connected to the dilution chamber 137.

The fluid moving member 180 is positioned on the upper portion of the fluid flow chamber 120 surrounded by the plurality of the reaction chambers 130. Holes 120a and 120b respectively corresponding to the first and second channels 210 and 220 of the valve 20 are formed at a bottom surface of the fluid flow chamber 120.

Accordingly, when the fluid moving member 180 moves up, a volume of the fluid flow chamber 120 is expanded and drawing power for suctioning the fluid to the inside of the fluid flow chamber 120 through the holes 120a and 120b is generated. When the fluid moving member 180 moves down, a volume of the fluid flow chamber 120 decreases and the fluid is discharged to the outside of the fluid flow chamber 120 through the holes 120a and 120b.

The holes 120a and 120b formed at the bottom surface of the fluid flow chamber 120 includes third holes 120a formed to correspond to the first outlet 212 of the first channel 210 and fourth holes 120b formed to correspond to the third outlet 222 of the second channel 220.

That is, the third holes 120a corresponds to the first outlet 212 of the first channel 210. Each of the first holes 130a formed at each of the reaction chambers 130 positioned at a concentric circle corresponds to the second outlet 214. In the plan view, the third holes 120a corresponding to the first holes 130a formed at the reaction chambers 130, respectively, are positioned on the diameter crossing a center of the housing 10.

That is, a third hole 121a is formed at the bottom surface of the fluid flow chamber 120 so that the third hole 121a is positioned on the same diameter of the first hole 131a of the binding chamber 131. A third hole 122a is formed at the bottom surface of the fluid flow chamber 120 so that the third hole 122a is positioned on the same diameter of the first hole 132a of the lysis chamber 132. Third holes 123a and 124a are formed at the bottom surface of the fluid flow chamber 120 so that the third holes 123a and 124a are positioned on the same diameter of the first holes 133a and 134a of the first and second washing chambers 133 and 134. A third hole 125a is formed at the bottom surface of the fluid flow chamber 120 so that the third hole 125a is positioned on the same diameter of the first hole 135a of the elution chamber 135. A third hole 127a is formed at the bottom surface of the fluid flow chamber 120 so that the third hole 127a is positioned on the same diameter of the first hole 137a of the dilution chamber 137.

However, the present invention is not limited thereto. It is sufficient that the first holes 130a corresponds to the second outlet 214 and the third holes 120a corresponds to the first outlet 212.

Also, the fourth holes 120b are closer to the central axis C than the third holes 120a to correspond to the third outlet 222. In this instance, a fourth hole 126b for a first PCR is positioned on the same diameter of a second hole 136b formed at the discard chamber 136, and a fourth hole 128b for a second PCR is not positioned on the same diameter of the other holes. The fourth hole 128b for the second PCR is a rotated position of the third hole 126b for the first PCR clockwise. This is because the usual rotation direction of the valve 20 is considered. However, the invention is not limited thereto. It is sufficient that the fourth holes 120b may correspond to the third outlet 212.

As described in the above, distances from the central axis C of the valve 20 to the first hole 130a, the second hole 136b, the third hole 120a, and the fourth hole 120b formed at the chamber 110 are different from each other. That is, the distance from the central axis C of the valve 20 to the first hole 130a is different from each of the distances from the central axis C of the valve 20 to the second hole 136b, the third hole 120a, and the fourth hole 120b.

Also, in a plan view, an imaginary line for connecting the first hole 130a and the third hole 120a, an imaginary line for connecting the second hole 136b and the center of the valve 20, and an imaginary line for connecting the fourth hole 120b and the center of the valve 20 may be not aligned with each other. Accordingly, a wanted chamber among the plurality of the chambers 110 can be selectively connected according to the rotation of the valve 20.

In the embodiment, the fluid flow chamber 120 having the fluid flow to each of the reaction chambers 130 exist is formed at the central portion, and thus, the path of the fluid flow can be minimized. Accordingly, the processing the sample and extracting the nucleic acid from sample can be smoothly performed.

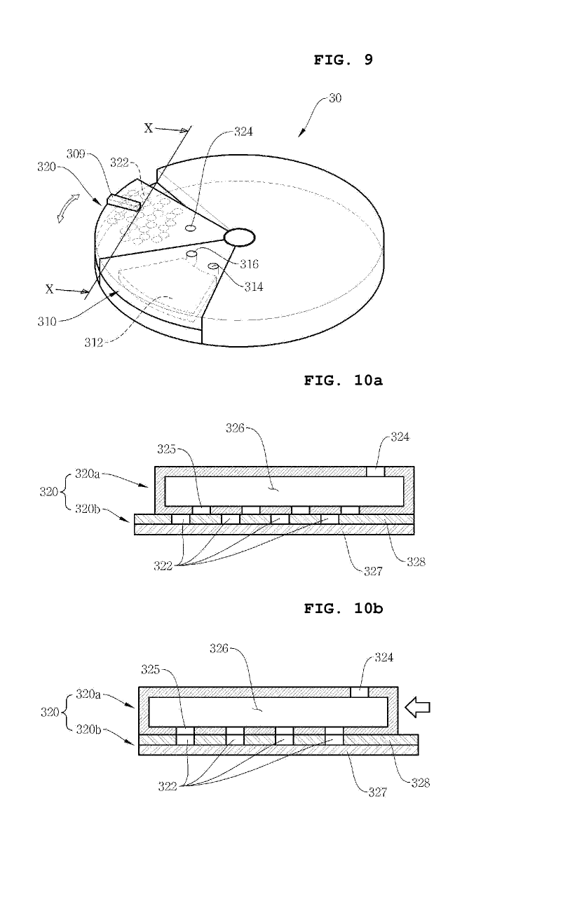

FIG. 9 is a perspective view of the PCR portion of the sample processing apparatus shown in FIG. 1, and FIG. 10a and FIG. 10b are cross-section views taken in a line of X-X of FIG. 9.

Referring to FIG. 9, as shown in the above, the PCR portion 30 includes the first PCR portion 310 and the second PCR portion 320 having the reaction spaces 312 and 322 having different shapes in the embodiment. Each of the first and second PCR portions 310 and 320 has a substantial fan shape, and the first and second PCR portions 310 and 320 have a radial shape to have a radial shape. In the drawing, it is exemplified that one first PCR portion 310 and one second PCR portion 320 are adjacent to each other and a space besides the one first PCR portion 310 and the one second PCR portion 320 is not used. However, the invention is not limited thereto. Therefore, the first PCR portion 310 may be separated from the second PCR portion 320, and the first PCR portion 310 may include a plurality of first PCR portions 310 and/or the second PCR portion 320 may include a plurality of second PCR portions 320. Various modifications are possible.

Accordingly, in the PCR portion 30 according to the embodiment, the first and the second PCR portions 310 and 320 are included so that the first PCR and the second PCR can be performed in sequence. Thereby, a NEST PCR can be performed. However, the invention is not limited to the NEST PCR. Thus the first PCR may be omitted.

In this instance, the first PCR may be a first-step PCR for the NEST PCR or a reverse transcription PCR (RT-PCR). Selectively, in the first PCR, the RT-PCR and the first-step PCR for the NEST PCR may be performed in sequence. The second PCR may be a second-step PCR of the NEST PCR or a usual PCR performed after the RT-PCR.

First, the case that the first PCR performed in the first PCR portion 310 is the first step of the NEST PCR and the second PCR performed in the second PCR portion 320 is the second step of the NEST PCR will be described. In the first PCR portion 310, the first PCR is performed by using a primer (for example, an outer primer) being complementarily bonded to an side outer than a target DNA. In the second PCR portion, the second PCR for obtaining the target DNA is performed by using a primer (for example, an inner primer).

The PCR process will be described in more detail.

The PCR amplifies the DNA by reacting a primer, a DNA polymerase, and deoxyribonucleoside triphosphate (“dNTP”), which is a material for forming new DNA, with the nucleic acid (particularly, the DNA).

In this instance, the PCR process may be classified into a denaturation process, an annealing, and an extension process.

In the denaturation process, the DNA polymerase separates double strands of the DNA to one stand to be used for a template. The denaturation process may be performed at a temperature of about 90˜96° C. The annealing process attaches the primer to the DNA separated to the single strand. The annealing process may be performed at a temperature of about 50˜65° C. When the temperature is excessively high, the primer and the DNA may be not bonded. When the temperature is excessively low, the primer may be not bonded complementary portions. That is, in the annealing process, the temperature is an important factor. The extension process extends and polymerizes the primer by the DNA polymerase and the dNTP. The extension process may be performed at a temperature suitable for the DNA polymerase. For example, the extension process may be performed at a temperature of 68˜74° C.

In the reaction spaces 312 and 322 of the first and second PCR portions 310 and 320, the primer, the DNA polymerase, the dNTP, and so on suitable for the first and second PCRs are positioned. Thereby, wanted first and second PCRs may be generated.

Next, the case that the first PCR performed in the first PCR portion 310 is the RT-PCR and the second PCR performed in the second PCR portion 320 is the usual PCR will be described. In the first PCR portion 310, the DNA is created by performing the RT-PCR to the collected RNA. In the second PCR portion 320, the wanted DNA can be amplified by using a primer. In RT-PCR, the DNA complementary to the RNA is creased at the predetermined temperature (for example, 40˜60° C.) by using a reverse transcriptase and the primer.

In this instance, in the reaction space 312 of the first PCR portion 310, the primer, the reverse transcriptase, and so on suitable for the RT-PCR are positioned to perform the RT-PCR. In the reaction space 322 of the second PCR portion 320, the primer, a DNA polymerase, and a dNTP suitable for the PCR are positioned to perform the PCR.

Finally, the case that the first PCR performed in the first PCR portion 310 is RT-PCR and a first PCR of the NEST PCR and the second PCR performed in the second PCR portion 320 is a second PCR of the NEST PCR will be described.

That is, the RT-PCR is performed to the collected DAN in order to create a DAN, and then, the first PCR is performed, in the first PCR portion 310. After that, the second PCR is performed to the DNA in the second PCR portion 320. In this instance, a primer, a reverse transcriptase, and so on suitable for the RT-PCR and a primer, a DNA polymerase, and a dNTP suitable for the first PCR are included in the reaction space 312 of the first PCR portion 310. Also, a primer, a DNA polymerase, and a dNTP suitable for the second PCR are included in the reaction space 322 of the second PCR portion 320.

In the first PCR portion 310, the RT-PCR is performed by providing a temperature condition suitable for the RT-PCR, and then, the first PCR is performed by providing a temperature condition suitable for the first PCR. After that, the second PCR is performed by providing a temperature condition suitable for the second PCR.

In the embodiment, since the PCR portion 30 includes the first and second PCR portions 310 and 320, the NEST PCR may performed, or the PCR after the RT-PCR may be performed. When the NEST PCR is performed, a nonspecific reaction can be reduced and accuracy or sensitivity can be enhanced by the PCR of two steps. When the PCR, along with the RT-PCR, is performed, it is applied to the case that the nucleic acid is the RNA, and thus, the RNA can be reverse-transcribed to the DNA and then the DNA can be amplified.

The first PCR and/or the RT-PCR are performed in the first PCR portion 310, and thus, the first PCR portion 310 should be heated to the suitable temperature. Therefore, the first PCR portion 310 has the single reaction space 312 having a small thickness and a large area. Then, a heat transfer can be easy, and the first PCR and/or the RT-PCR can be smoothly generated.

An exhaust hole 316 is additionally formed at an upper surface of the first PCR portion 310, as well as the outlet 314. In this instance, the outlet 314 is connected to the fourth outlet 224 of the second channel 220 and thus is connected to the fluid flow chamber (reference numeral 120 FIG. 4). The exhaust hole 316 is connected the sixth outlet 234 of the third channel 230 and thus is connected to the discard chamber 136. Since the first PCR portion 310 has the single reaction space 312 having a large area, if only the outlet 314 is included, the material may not be transferred when the elution buffer including the nucleic acid positioned in the fluid flow chamber 120 is injected into the first PCR portion 310 or a material after the first PCR is discharged. In the embodiment, the exhaust hole 316 is included as well as the outlet 314, when the material including the nucleic acid is injected to or is discharged from the outlet 314, the air flows out to the discard chamber 136 through the exhaust hole 316 or the air of the discard chamber 136 flows into the first PCR portion 310. Accordingly, the injection and the discharge of the material, the material can smoothly flow.

For example, a distance from the outlet 314 and the central axis C of the PCR portion 30 is the same as a distance from the exhaust hole 316 and the central axis C of the PCR portion 30. However, the invention is not limited thereto. It is sufficient that the outlet 314 corresponds to the fourth outlet 224 and the exhaust hole 316 corresponds to the sixth outlet 234.

The second PCR portion 320 may include a plurality of the reaction spaces 322 for performing the second PCR. This is for a multiple diagnoses at the same time. By using primers corresponding to a plurality of base sequences, respectively, amplification can be generated at the same time, and thus, existence and nonexistence of a plurality of target DNAs can be determined. For example, when the primes for amplifying the target DNAs of various respiratory diseases are put into the reaction spaces, respectively, and the target DNAs are amplified. Specific amplifying is generated only at the reaction space where the target DNA exists and the target DNA is detected. Thus, multiple diseases can be diagnosed. Also, a deletion variant of dystrophin gene, which is a reason of myopathy, can be detected by 18 kinds of primers. By having at least 18 reaction spaces 322, the deletion variant of the dystrophin gene can be detected. Each of the reaction spaces 322 may have a recess shape, and the reaction spaces 322 may be spaced from each other.

The outlet 324 is formed at the upper surface of the second PCR portion 320. In this instance, the outlet 324 is connected to the fourth outlet 224 of the second channel 220 and thus is connected to the fluid flow chamber (reference numeral 120 of FIG. 4).

More particularly, the second PCR portion 320 includes a first portion 320a and a second portion 320b. Thus, when the diluted mixture is supplied to the second PCR portion 320, the primers positioned at the plurality of the reaction space 322 are prevented from being mixed to each other.

The outlet 324 is formed at the upper surface of the first portion 320a and channels 325 are formed at the lower surface of first portion 320a. A fluid-inflowing space 326 for receiving the diluted mixture is formed at an inside of the first portion 320a. The barrier 328 is formed at the second portion 320b to surround the reaction spaces 322 so that the reaction spaces 322 are formed on the supporting member 327. The supporting member 327 may include a transparent material (for example, a transparent film). The barrier 328 may include a sealant material including silicone and the like. An arrangement of the channels 325 and an arrangement of the reaction spaces 322 are the same. In a normal state, the channels 325 are slightly deviated from the reaction spaces 322. When the first portion 320a moves, the channels 325 coincide with the reaction spaces 322, respectively.

That is, as shown in FIG. 10a, in the normal state, when the diluted mixture is supplied to the fluid-inflowing space 326 through the outlet 324, the channels 325 of the first portion 320a are positioned on the barrier 328 of the second portion 320b, and the diluted mixture is positioned in the fluid-inflowing space 326. After the completion of supplying the diluted mixture, as shown in FIG. 10b, the first portion 320a moves and the channels 325 and the reaction space 322 of the second portion 320b are connected to each other. Then, the diluted mixture of the fluid-inflowing space 326 can be injected to each of the reaction spaces 322. The first portion 320a may be transferred by a driving member (not shown) for driving the first portion 320a and included in the automatic analyzing apparatus 400.

In this instance, the first portion 320a may be rotated by the PCR moving portion 209 formed at the lower surface of the valve 20 and the engaged portion 329 formed at the upper surface of the PCR portion 30. That is, as the valve 20 rotates, the PCR moving portion 209 of the valve 20 is engaged with the coupled portion 32 of the PCR portion 30. In this state, if the valve 20 rotates more, the PCR moving portion 209 pushes the engaged portion 329, and thereby rotating the first portion 320a. However, this is an example for moving the first portion 320a, and various modifications are possible. Not shown in the drawings, a groove or a recess portion is formed at the lower surface of the valve 20 to correspond to a path where the engaged portion 329 of the PCR portion 30 moves. Thereby, the PCR portion 30 is in close contact with the valve 20 even the engaged portion 329 is formed at the upper surface of the PCR portion 30.

By the structure of the second PCR portion 320, the primers of the reaction spaces 322 are prevented from being mixed with each other when the diluted mixture is supplied and goes through the reaction spaces 322.

Hereinafter, an automatic analyzing apparatus will be described. The automatic analyzing apparatus includes the sample processing apparatus 100 having the housing 10, the valve 20, and the PCR portion 30, and can automatically perform a process of obtaining the target DNA from the sample and a process of detecting the pathogen of the target DNA.

FIG. 11 is a perspective view of an automatic analyzing apparatus according to an embodiment of the invention, and FIG. 12 is a schematically cross-sectional view of the automatic analyzing apparatus shown in FIG. 11.

As shown in FIG. 11, an automatic analyzing apparatus 400 according to an embodiment includes an apparatus portion. In the apparatus portion, the sample processing apparatus 100 is installed. The apparatus portion obtains the target DNA from the sample by driving the sample processing apparatus 100 and the detecting member 450 detects a pathogen. This will be described in more detail.

Referring to FIG. 12, the automatic analyzing apparatus 400 includes an up-down driving member (not shown), a rotation driving member 410, an ultrasonic-driving driving member 430, a heating member 440, and a detecting member 450. The up-down driving member moves the fluid moving member 180 up and down. The rotation driving member 410 rotates the valve 20. The ultrasonic-member driving member 430 drives the ultrasonic member 190. The heating member 440 heats the PCR portion 30. The detecting member 450 detects a pathogen.

Various methods and structure being able to move the fluid moving member 180 up and down may be applied to the up-down driving member. Various methods and structure being able to rotate the valve 20 clockwise and/or counterclockwise may be applied to the rotation driving member 410. For example, the rotation driving member 410 may be a stepper motor. Various methods and structure supplying energy to generate the ultrasonic waves at the ultrasonic member 190 may be applied to the ultrasonic-member driving member 430.

Various methods and structure being able to heat the PCR portion 30 to a wanted temperature may be applied to the heating member 440. For example, the heating member 440 may include a surface type heater.

Various methods and structure being able to discriminate the existence and nonexistence of the target DNA amplified by the primer in the second PCR portion 320 may be applied to the detecting member 450. For example, in the embodiment, the detecting member 450 includes a light source 452 providing light to the second PCR portion 320 and a camera taking a photograph of the second PCR portion 320 when the light is provided by the light source 452.

Known various elements, devices, or apparatuses may be used for the up-down driving member, the rotation driving member 410, the ultrasonic-member driving member 430, the heating member 440, and the detecting member 450.

Also, in the embodiment, it is exemplified that the detecting member 450 and the heating member 440 are positioned at opposite sides to each other with respect to the PCR portion 30. However, the invention is not limited thereto. When the heating member 440 is transparent, the detecting member 450 and the heating member 440 may be positioned at the same side with respect to the PCR portion 30. The heating member 440 may be positioned at both sides of the PCR portion 30. Also, a heating type using fluid such as hot air or liquid may be applied to the heating member 440, instead of the surface type heater. That is, the PCR portion 30 may be heated by using the hot air or liquid.

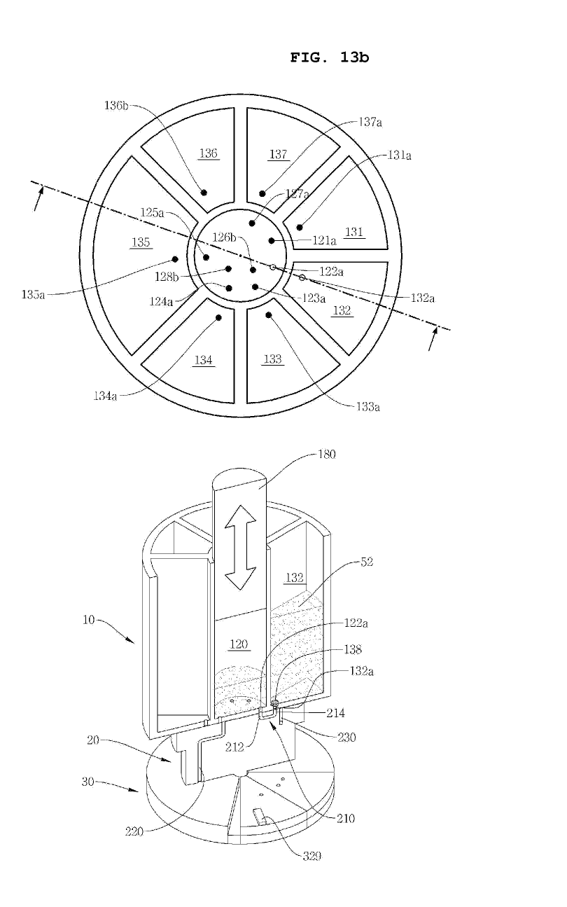

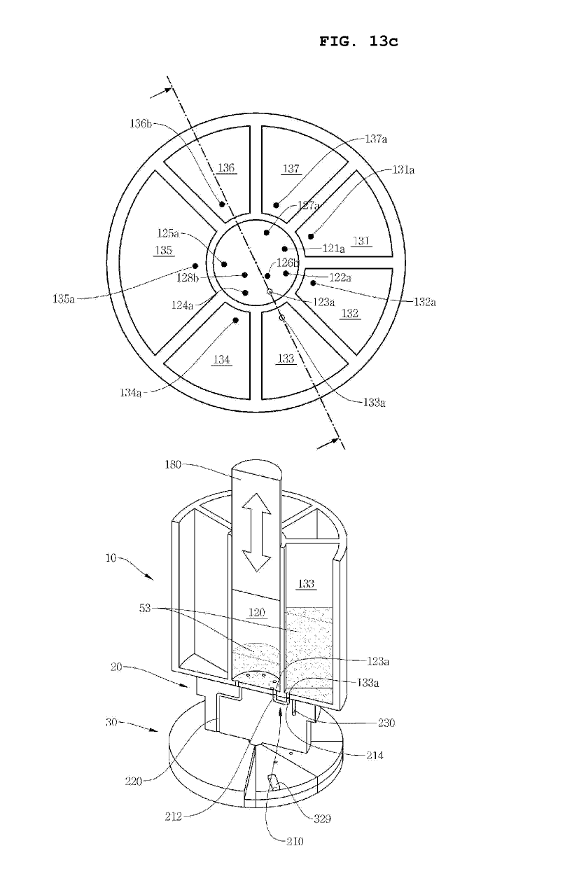

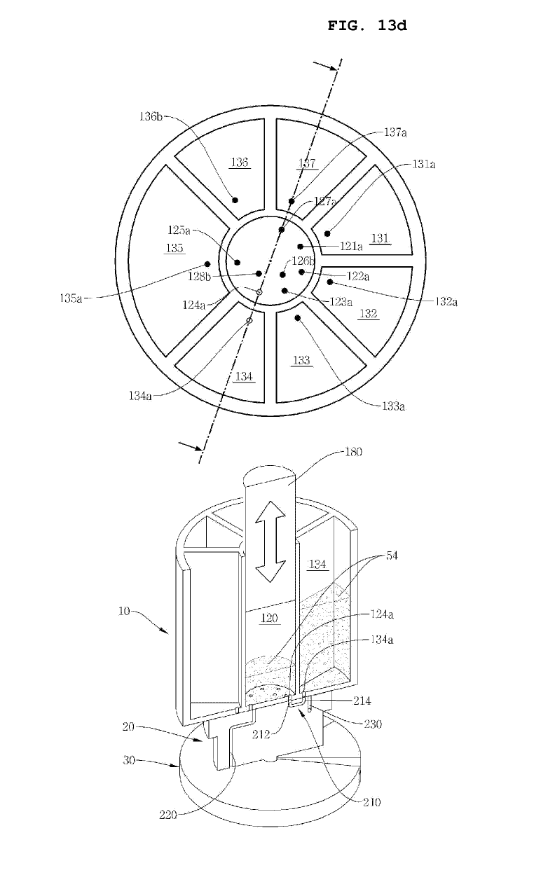

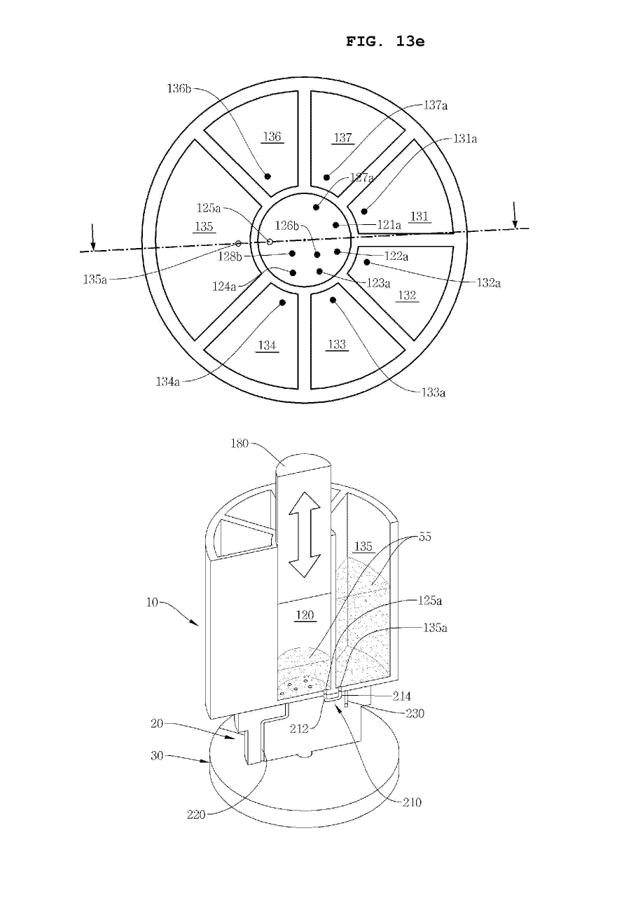

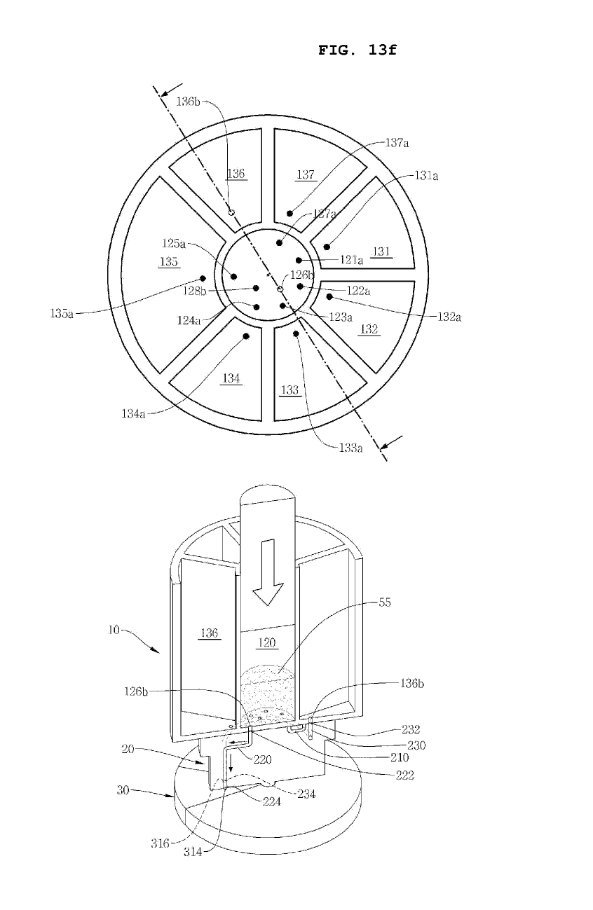

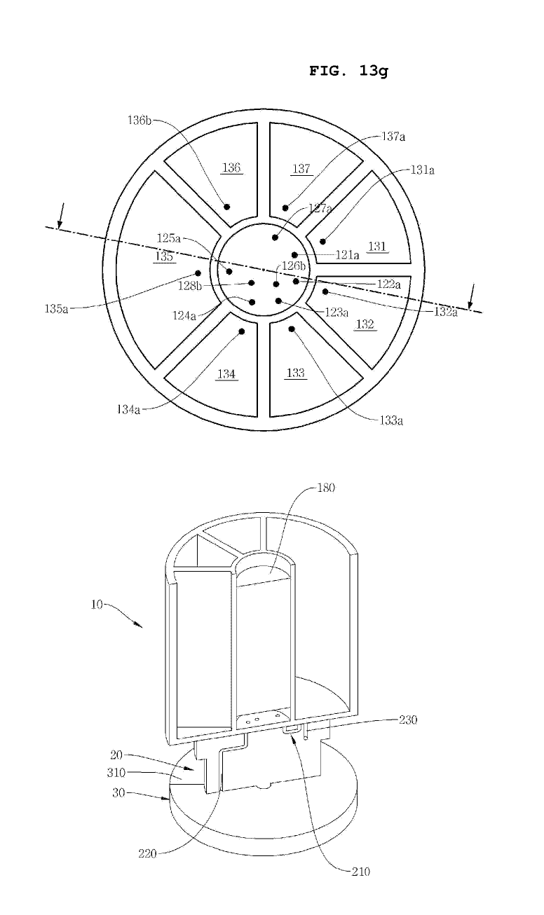

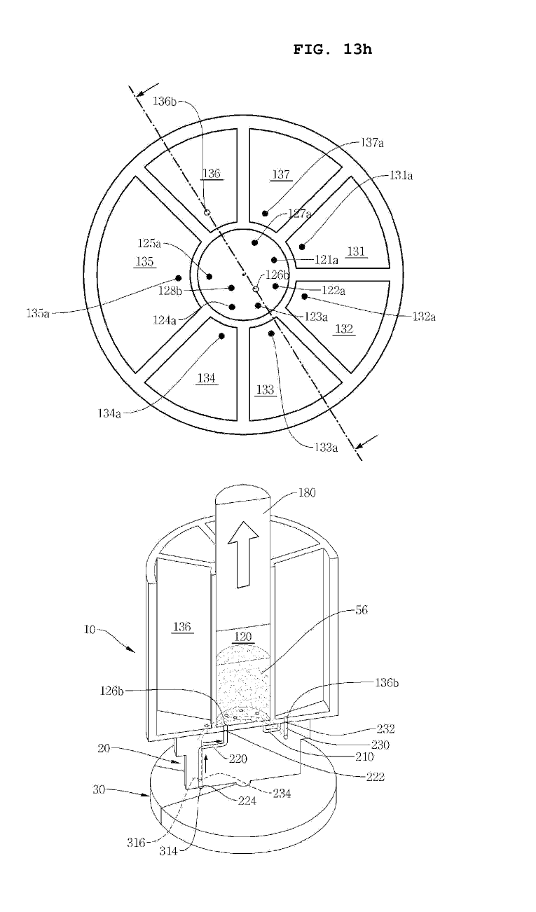

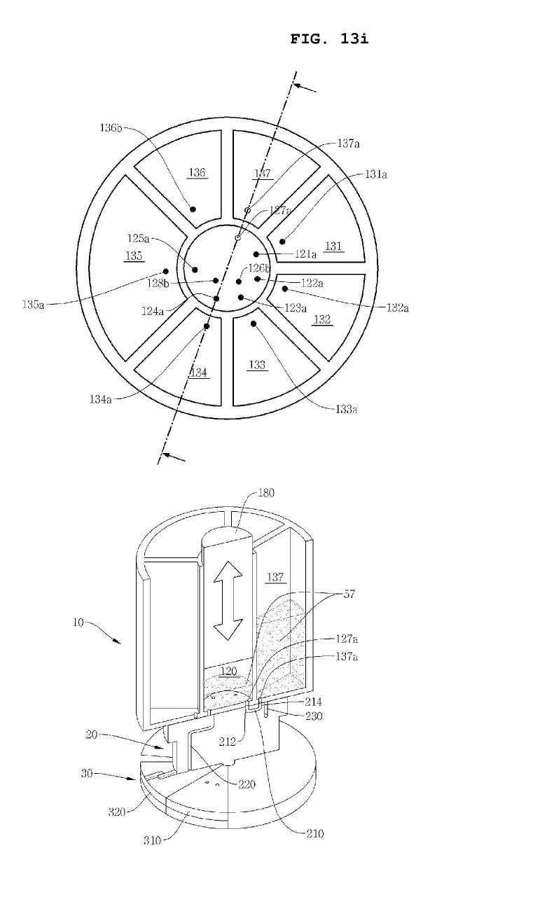

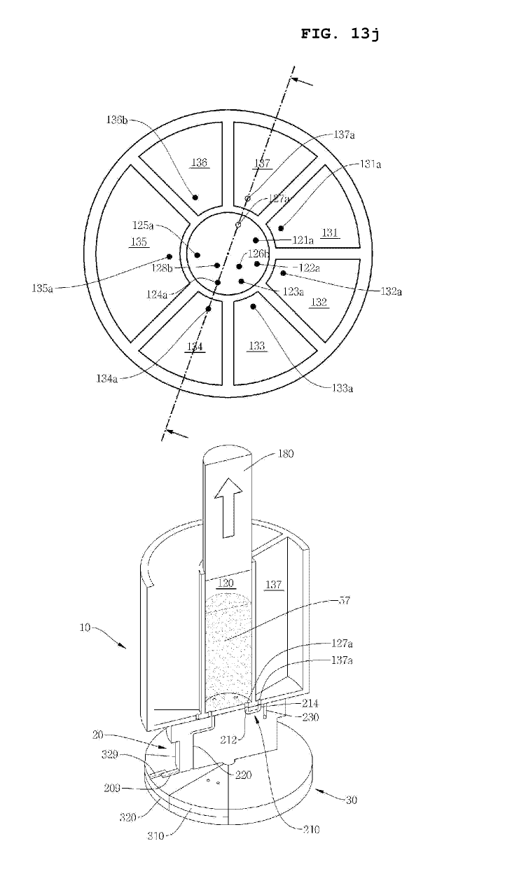

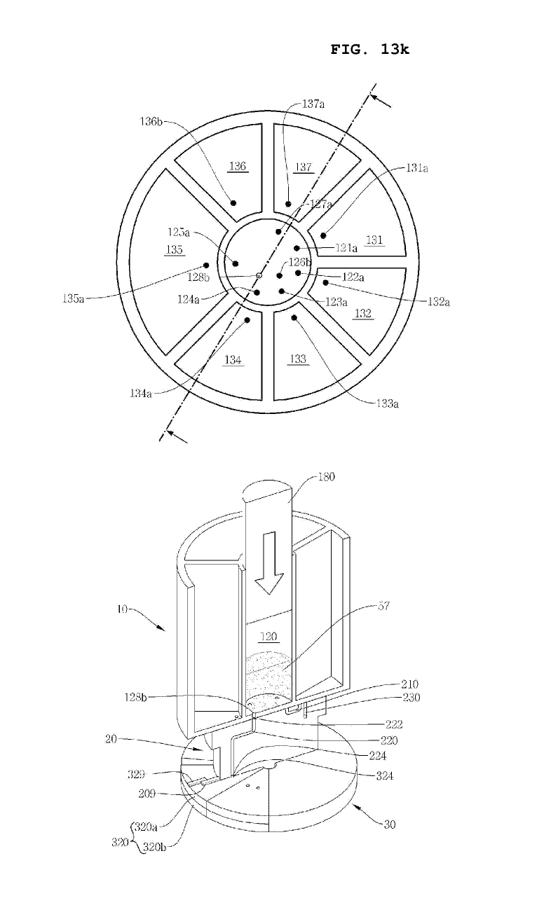

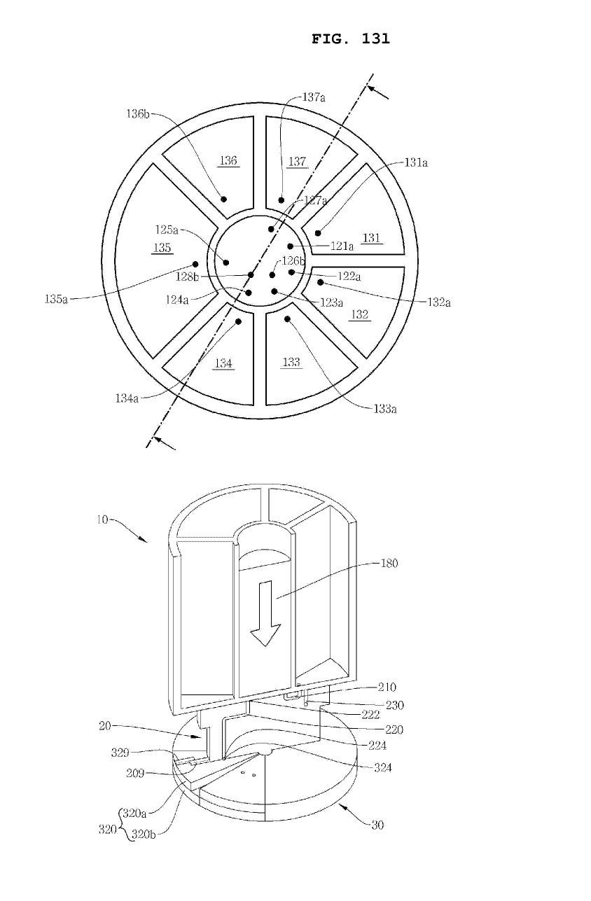

Hereinafter, with reference to FIG. 13a to FIG. 13l, an operation of the automatic analyzing apparatus 400 including the sample processing apparatus 100 will be described in detail. FIG. 13a to FIG. 13l are views for describing an operation of the sample processing apparatus according to the embodiment. Plan views of the housing are shown at upper portions of FIG. 13a to FIG. 13l, and sectional perspective views of the sample processing apparatus 100 taken in dotted lines of the plan views are shown at lower portions of FIG. 13a to FIG. 13l. For clear descriptions, the cover portion 150 is omitted in the sectional perspective views.

First, the sample is put into the lysis chamber 132, and the lysis reaction is generated. In this instance, as shown in FIG. 13a, the second outlet 214 of the first channel 210 is connected to the first hole 131a of the binding chamber 131, and the first outlet 212 of the first channel 210 is connected to the third hole 121a of the fluid flow chamber 120. The second channel 220 and the third channel 230 are not connected to the fluid flow chamber 120, the reaction chamber 130, and the PCR portion 30.

In this state, the fluid moving member 180 repeatedly moves up and down, and a binding buffer 51 of the binding chamber 131 repeatedly flows through the first channel 210. Then, the binding buffer 51 inside the first channel 210 is introduced to the first filter (reference numeral 216 of FIG. 4). And then, the fluid moving member 180 moves down, and thus, the binding buffer 51 moves to the inside of the binding chamber 131.

When the lysis reaction is induced in the lysis chamber 132, the binding buffer 51 is positioned in the first channel 210 during the lysis reaction by connecting the first channel 210 to the binding chamber 131. Accordingly, a step for rotating the valve 20 in order to introduce the binding buffer 51 to the first channel 210 after the lysis reaction is not necessary. Thus, the process can be simplified.

Next, as shown in FIG. 13b, by rotating the valve 20, the second outlet 214 of the first channel 210 is connected to the first hole 132a of the lysis chamber 132, and the first outlet 212 of the first channel 210 is connected to the third hole 122a of the fluid flow chamber 120. The second channel 220 and the third channel 230 are not connected to the fluid flow chamber 120, the reaction chamber 130, and the PCR portion 30.

In this state, by repeatedly moving the fluid moving member 180 up and down, a lysis buffer 52 where the nucleic acid is dissolved repeatedly penetrates through the first filter 216 of the first channel 210, and thereby, collecting the nucleic acid at the first filter 216. The cell debris in the lysis chamber 132 is filtered by the second filter 138 and thus cannot flow into the first channel 210. After that, by pushing the fluid moving member 180 down, the materials remained in the fluid flow chamber 120 moves to the lysis chamber 132 and are discarded. Here, it is exemplified that the fluid moving member 180 repeatedly moves up and down. However, the invention is not limited thereto, and the fluid moving member 180 may move up and down just one time. This will be applied to the following descriptions.

Next, as shown in FIG. 13c, by rotating the valve 20, the second outlet 214 of the first channel 210 is connected to the first hole 133a of the first washing chamber 133, and the first outlet 212 of the first channel 210 is connected to the third hole 123a of the fluid flow chamber 120. The second channel 220 and the third channel 230 are not connected to the fluid flow chamber 120, the reaction chamber 130, and the PCR portion 30.

In this state, by repeatedly moving the fluid moving member 180 up and down, the first washing buffer 53 in the first washing chamber 133 repeatedly penetrates through the first filter 216 of the first channel 210. Thereby, the first filter 216 is washed. After that, by pushing the fluid moving member 180 down, the materials remained in the fluid flow chamber 120 moves to the first washing chamber 133 and are discarded.

Next, as shown in 13d, by rotating the valve 20, the second outlet 214 of the first channel 210 is connected to the first hole 134a of the second washing chamber 134, and the first outlet 212 of the first channel 210 is connected to the third hole 124a of the fluid flow chamber 120. The second channel 220 and the third channel 230 are not connected to the fluid flow chamber 120, the reaction chamber 130, and the PCR portion 30.

In this state, by repeatedly moving the fluid moving member 180 up and down, the second washing buffer 54 in the second washing chamber 134 repeatedly penetrates through the first filter 216 of the first channel 210. Thereby, the first washing buffer 53 is eliminated and the first filter 216 is washed. After that, by pushing the fluid moving member 180 down, the materials remained in the fluid flow chamber 120 moves to the second washing chamber 134 and are discarded.

Next, as shown in FIG. 13e, by rotating the valve 20, the second outlet 214 of the first channel 210 is connected to the first hole 135a of the elution chamber 135, and the first outlet 212 of the first channel 210 is connected to the third hole 125a of the fluid flow chamber 120. The second channel 220 and the third channel 230 are not connected to the fluid flow chamber 120, the reaction chamber 130, and the PCR portion 30.

In this state, by repeatedly moving the fluid moving member 180 up and down, an elution buffer 55 in the elution chamber 135 repeatedly penetrates through the first filter 216 of the first channel 210. Thereby, the nucleic acid at the first filter 216 is dissolved. After that, the elution buffer 55 where the nucleic acid is dissolved is introduced to the fluid flow chamber 120. In this instance, an amount of the elution buffer 55 is controlled to be suitable with consideration of the reaction space 312 of the first PCR portion 310.

Next, as shown in FIG. 13f, by rotating the valve 20, the third outlet 222 of the second channel 220 is connected to the third hole 126b for the first PCR of the fluid flow chamber 120, and the fourth outlet 224 of the second channel 230 is connected to the outlet 314 of the first PCR portion 310. The fifth outlet 232 of the third channel 220 is connected to the second hole 136b for the discard chamber 136, and the sixth outlet 234 of the third channel 230 is connected to the exhaust hole 316 of the first PCR portion 310. The first channel 210 is not connected to the reaction chamber 130 and the PCR portion 30.

In this state, by moving the fluid moving member 180 down, the elution buffer 55 where the nucleic acid is dissolved is introduced into the reaction space 312 of the first PCR portion 310.

Next, as shown in FIG. 13g, by rotating the valve 20 slightly, the first to sixth outlets 212, 214, 222, 224, 232, and 234 of the first to third channels 210, 220, and 230 are closed, and the first PCR is performed in the first PCR portion 310. The heating member (reference numeral 440 of FIG. 12) of the automatic analyzing apparatus 400 heats the first PCR portion 310 to a temperature suitable for the first PCR. In this state, the amplifying process of the heating and cooling is performed and completed.

Next, as shown in FIG. 13h, by rotating the valve 20, the third outlet 222 of the second channel 220 is connected to the third hole 126b for the first PCR of the fluid flow chamber 120, and the fourth outlet 224 of the second channel 230 is connected to the outlet 314 of the first PCR portion 310. The fifth outlet 232 of the third channel 220 is connected to the second hole 136b for the discard chamber 136, and the sixth outlet 234 of the third channel 230 is connected to the exhaust hole 316 of the first PCR portion 310. The first channel 210 is not connected to the fluid flow chamber 120 and the reaction chamber 130.

In this state, by moving the fluid moving member 180 up, a first treated material 56 that the first PCR is complete is introduced to the fluid flow chamber 120.

Next, as shown in FIG. 13i, by rotating the valve 20, the second outlet 214 of the first channel 210 is connected to the first hole 137a of the dilution chamber 137, and the first outlet 212 of the first channel 210 is connected to the third hole 127a of the fluid flow chamber 120. The second channel 220 and the third channel 230 are not connected to the fluid flow chamber 120, the reaction chamber 130, and the PCR portion 30.

In this state, by repeatedly moving the fluid moving member 180 up and down, a diluted mixture 57 is formed by mixing a dilution buffer of the dilution chamber 137 and the first treated material 56 of the fluid flow chamber 120. An amount of the dilution buffer is selected to consider an amount of the elution buffer, a volume of the reaction space 322 of the second PCR portion 320.

Next, as shown in FIG. 13j, by moving the fluid moving member 180 up, the diluted mixture 57 is introduced into the fluid flow chamber 120.

Next, as shown in FIG. 13k, by rotating the valve 20, the third outlet 222 of the second channel 220 is connected to the third hole 128b for the second PCR of the fluid flow chamber 120, and the fourth outlet 224 of the second channel 230 is connected to the outlet 324 of the second PCR portion 320. The first channel 210 and the third channel 230 are not connected to the fluid flow chamber 120, the reaction chamber 130, and the PCR portion 30.

In this state, by moving the fluid moving member 180 down, the diluted mixture 57 is introduced to the reaction space 322 of the second PCR portion 320. As stated in the above, in the state shown in FIG. 10a, the diluted mixture 57 is supplied to the second PCR portion 320, and is supplied to the fluid-inflowing space 326. Next, the PCR moving portion 209 of the valve 20 is engaged with the engaged portion 329 by rotating the valve 20, and then, only the first portion 320a of the second PCR portion 320 moves more by rotating the valve 20. Then, as shown in FIG. 13l and FIG. 10b, the first portion 320a moves and the diluted mixture 57 of the fluid-inflowing space 326 is supplied to the reaction spaces 322 through the channels 325. After that, reversely rotating the valve 20, the reaction spaces 322 are closed. After closing the reaction spaces 322, a process of eliminating the diluted mixture 57 that may be remained in the fluid-inflowing space 326 may be selectively performed. (Various methods may be used for the method for the diluted mixture 57, and the invention is not limited thereto. For example, the valve 20 is reversely rotated, and then, the fourth outlet 224 of the second channel 220 and the outlet 324 of the second PCR portion 320 are connected to each other. Accordingly, the remained diluted mixture 57 can be transferred to the fluid flow chamber 120 by rising the fluid moving member 180.) After that, the heating member 440 of the automatic analyzing apparatus 400 heats the second PCR portion 320 to a temperature suitable for the second PCR, and thereby, performing the second PCR.

When the second PCR is complete or the second PCR is performed (that is, a real time PCR), a photograph of the second PCR portion 320 is taken by the detecting member (reference numeral 450 of FIG. 12)(that is, the light source (reference numeral 452 of FIG. 12) and the camera (reference numeral 454 of FIG. 12)). The existence and nonexistence of the pathogen can be determined by a photo interpretation. A method for discriminate disease is well known through an analysis of data optically obtained, and thus, the detailed descriptions will be omitted.

As in the above, in the sample processing apparatus 100 according to the embodiment, by the rotation the valve 20 including the plurality of channels 210, 220, and 230 that are not connected to each other, the chambers 110 of the housing 10 are connected to each other or the fluid flow chamber 120 and the PCR portion 30 are connected to each other. Thus, processes of extracting the nucleic acid and amplifying the nucleic acid can be automatically performed in sequence. In this instance, the PCR portion 30 includes the first and the second PCR portions 310 and 320, and therefore, the accuracy of the PCR can be enhanced. Also, the automatic analyzing apparatus 400 including the sample processing apparatus 100 can automatically determine the existence and nonexistence of the pathogen from the target DNA generated by the sample processing apparatus 100. Further, the automatic analyzing apparatus 400 including the sample processing apparatus 100 can detect multiple pathogens at the same time.

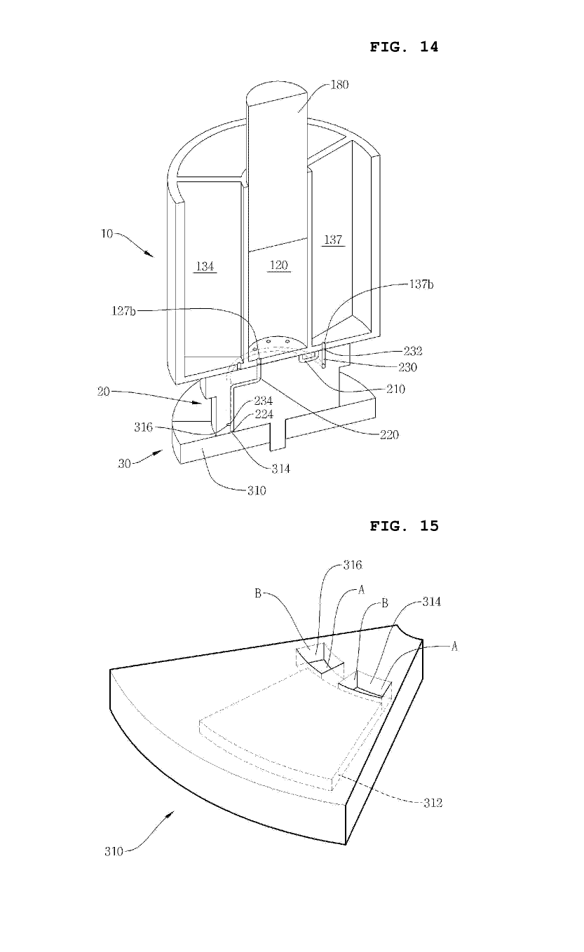

Hereinafter, with reference to FIG. 14 and FIG. 15, a modified embodiment of the invention will be described in more detail. The descriptions for the portions that are the same as or similar to the above embodiment will be omitted, and the other portions will be described in detail.

FIG. 14 is a sectional perspective view of a housing of an automatic analyzing apparatus according to a modified embodiment, and FIG. 15 is a perspective view of a PCR portion of the automatic analyzing apparatus according to the modified embodiment.

Referring to FIG. 14, in the embodiment, a dilution chamber 137 includes a second hole 137b corresponding to the fifth outlet 232 of the third channel 230, instead the first hole 137a corresponding to the second outlet 214 of the first channel 210. Also, the fluid flow chamber 120 includes a fourth hole 127b corresponding to the third outlet 222 of the second channel 220, instead the third hole 127a corresponding to the first outlet 212 of the first channel 210. Referring to FIG. 15, in the PCR portion 30 of the embodiment, the outlet 314 and the exhaust hole 316 of the first PCR portion 310 longitudinally extended along paths of the fourth outlet 224 and the sixth outlet 234.

In the automatic analyzing apparatus, steps from the first process to the process of eluting the nucleic acid at the first filter 216 by using the elution buffer 55 of the elution chamber 135 are the same as those of the above embodiment (particularly, FIG. 13a to FIG. 13e, and the detailed description thereof).

In a step for introducing the elution buffer where the nucleic acid is dissolved to the first PCR portion 310 (refer to FIG. 13f), the second channel 220 and the third channel 230 are connected to one end of the outlet 314 and one end of the exhaust hole 316 of first PCR portion 310 (for example, A portions of drawings), respectively. The step of performing the first PCR (refer to FIG. 13i and the detailed descriptions thereof) is the same as the above embodiment.

And then, by rotating the valve 20, the third outlet 222 of the second channel 220 coincides with the fourth hole 127b of the fluid flow chamber 120, and the fourth outlet 224 coincides with the other end of the outlet 314 of the first PCR portion 310 (B portion of drawings). At the same time, the fifth outlet 232 of the third channel 230 coincides with the second hole 137b of the dilution chamber 137, and the sixth outlet 234 coincides with the other end of the exhaust hole 316 of the first PCR portion 310 (B portion of drawings). In this state, by moving the fluid moving member 180, the dilution buffer of the dilution chamber 137 penetrates through the first PCR portion 310 and then moves to the fluid flow chamber 120. Then, the diluted mixture (reference numeral 57 of FIG. 13i) where the first treated material formed by the first PCR in the first PCR portion 310 and the dilution buffer are mixed to each other flows into the fluid flow chamber 120.

And then, by rotating the valve 20, the diluted mixture 57 is introduced to the second PCR portion 320, and the second PCR is performed (refer to FIG. 13k and the detailed descriptions thereof).

The above described features, configurations, effects, and the like are included in at least one of the embodiments of the present invention, and should not be limited to only one embodiment. In addition, the features, configurations, effects, and the like as illustrated in each embodiment may be implemented with regard to other embodiments as they are combined with one another or modified by those skilled in the art. Thus, content related to these combinations and modifications should be construed as including in the scope and spirit of the invention as disclosed in the accompanying claims.

### INDUSTRIAL APPLICABILITY

A sample processing apparatus according to an embodiment of the invention can automatically perform several processes necessary to process a sample and to detect a pathogen, and an automatic analyzing apparatus includes the sample processing apparatus. The sample processing apparatus and an automatic analyzing apparatus are industrially applicable.

1. A sample processing apparatus for extracting a nucleic acid from a sample and amplifying the nucleic acid, wherein the sample processing apparatus comprising:
a housing comprising a chamber;
a valve positioned below the housing; and
a polymerase chain reaction (PCR) portion positioned below the valve, wherein the polymerase chain reaction portion performing a polymerase chain reaction.

2. The sample processing apparatus according to claim 1, wherein the housing and the polymerase chain reaction portion are coupled while interposing the valve, and
the valve is rotatably coupled.

3. The sample processing apparatus according to claim 1, wherein the housing comprises a plurality of extended portions extended downward,
each of the plurality of extended portions comprises a first coupled portion, and
the polymerase chain reaction portion comprises a second coupled portion coupled to the first coupled portion.

4. The sample processing apparatus according to claim 3, wherein the first coupled portion comprises a hole or a recess, and
the second coupled portion comprises a protruded portion inserted into the hole.

5. The sample processing apparatus according to claim 1, wherein a planar shape of each of the housing, the valve, and the polymerase chain reaction portion is a circular shape.

6. The sample processing apparatus according to claim 1, wherein an area of a lower surface of the valve is smaller than an area of an upper surface of the valve.

7. The sample processing apparatus according to claim 1, wherein the valve comprises an upper portion having a first diameter in a plan view and a lower portion having a second diameter smaller than the first diameter in the plan view.

8. The sample processing apparatus according to claim 6, wherein the valve comprises a channel for a fluid flow inside the valve,
the chamber comprises a hole connected to the channel on a bottom surface of the chamber, and
the polymerase chain reaction portion comprises an outlet connected to the channel at an upper surface of the polymerase chain reaction portion.

9. The sample processing apparatus according to claim 1, further comprising:
a cover portion for covering the housing.

10. The sample processing apparatus according to claim 9, wherein the cover portion comprises:
a first cover portion fixed to an upper edge of the housing and comprising an opening to correspond to the chamber; and
a second cover portion fixed on the first cover portion to cover the opening.

11. The sample processing apparatus according to claim 10, wherein a part of the first cover portion and a part of the second cover portion are foldably connected to each other.

12. An automatic analyzing apparatus, comprising:
a sample processing apparatus for extracting a nucleic acid from a sample and amplifying the nucleic acid; and
an apparatus portion where the sample processing apparatus is installed, wherein apparatus portion comprising a driving member for driving the sample processing apparatus, a heating member for heating the sample processing apparatus, and a detecting member for determining existence and nonexistence of a pathogen from the nucleic acid amplified from the sample processing apparatus,
wherein the sample processing apparatus comprises:
a housing comprising a chamber;
a valve positioned below the housing; and
a polymerase chain reaction (PCR) portion positioned below the valve, wherein the polymerase chain reaction portion performing a polymerase chain reaction.

13. The automatic analyzing apparatus according to claim 12, wherein the housing and the polymerase chain reaction portion are coupled while interposing the valve, and
the valve is rotatably coupled.

14. The automatic analyzing apparatus according to claim 12, wherein the housing comprises a plurality of extended portions extended downward,
each of the plurality of extended portions comprises a first coupled portion, and
the polymerase chain reaction portion comprises a second coupled portion coupled to the first coupled portion.

15. The automatic analyzing apparatus according to claim 12, wherein a planar shape of each of the housing, the valve, and the polymerase chain reaction portion is a circular shape.

16. The automatic analyzing apparatus according to claim 12, wherein an area of a lower surface of the valve is smaller than an area of an upper surface of the valve.

17. The automatic analyzing apparatus according to claim 12, wherein the valve comprises a channel for a fluid flow inside the valve,
the chamber comprises a hole connected to the channel on a bottom surface of the chamber, and
the polymerase chain reaction portion comprises an outlet connected to the channel at an upper surface of the polymerase chain reaction portion.

18. The automatic analyzing apparatus according to claim 12, further comprising:
a cover portion for covering the housing.

19. The automatic analyzing apparatus according to claim 18, wherein the cover portion comprises:
a first cover portion fixed to an upper edge of the housing and comprising an opening to correspond to the chamber; and
a second cover portion fixed on the first cover portion to cover the opening.

20. The automatic analyzing apparatus according to claim 19, wherein a part of the first cover portion and a part of the second cover portion are foldably connected to each other.
